(19) United States
(12) Patent Application Publication
(10) Pub. No.: US 2015/0010639 A1
(43) Pub. Date: Jan. 8, 2015

(54) PHARMACEUTICAL OR DIETARY COMPOSITIONS BASED ON SHORT-CHAIN FATTY ACIDS AND COMPLEX SUGARS, FOR INTESTINAL DISORDERS

(71) Applicant: COSMO TECHNOLOGIES LTD., Dublin (IE)

(72) Inventors: Mauro AJANI, Lainate (Milano) (IT); Roberto VILLA, Lainate (Milano) (IT); Giuseppe CELASCO, Lainate (Milano) (IT); Luigi MORO, Lainate (Milano) (IT)

(73) Assignee: COSMO TECHNOLOGIES LTD., Dublin (IE)

(21) Appl. No.: 14/496,837

(22) Filed: Sep. 25, 2014

Related U.S. Application Data
(63) Continuation of application No. 10/588,272, filed on Aug. 4, 2006, now abandoned.
(60) Provisional application No. 60/553,549, filed on Mar. 17, 2004.

(30) Foreign Application Priority Data
Feb. 6, 2004 (IT) MI2004 A 000187

(51) Int. Cl. A23L 1/30; A61K 9/28; A61K 31/716; A61K 31/733; A23L 1/308; A61K 31/732

(52) U.S. Cl. CPC A23L 1/3008; A23L 1/308; A61K 31/732; A61K 31/716; A61K 31/733; A61K 9/28; A23V 2002/00
USPC 424/490; 426/601; 426/89; 426/103; 426/2; 514/54; 514/58

(57) ABSTRACT

Pharmaceutical or dietary composition containing, as active ingredients, at least one short-chain fatty acid or salt, ester and/or amide thereof, in combination with a complex sugar and/or dietary fibre. The complex sugar and/or dietary fibre is selected from inulin, pectin, dextrin, maltodextrin or derivatives thereof and with one or more pharmacologically acceptable excipients. The composition has (a) a matrix consisting of lipophilic compounds with a melting point lower than 90° C. and optionally amphiphilic compounds in which the active ingredient are at least partially incorporated, (b) an amphiphilic matrix; and (c) an outer hydrophilic matrix in which the lipophilic matrix and the amphiphilic matrix are dispersed.

### CROSS-REFERENCE TO RELATED APPLICATIONS

The present application is a continuation of U.S. patent application Ser. No. 10/588,272, filed 4 Aug. 2006, which is a 35 U.S.C. §371 National Phase Entry Application of PCT/EP05/50414, filed 1 Feb. 2005, and designating the United States, which in turn claims priority to Italian Patent Application No. MI2004 A 000187, filed 6 Feb. 2004, and U.S. Provisional Patent Application Ser. No. 60/553,549, filed 17 Mar. 2004. Each application is incorporated herein by reference.

### BACKGROUND OF THE INVENTION

Short-chain fatty acids (SCFA) are linear or branched C₁-C₅ monocarboxylic organic acids such as acetic, propionic, butyric, and isovaleric acids.

They are produced by the fermentation of undigested sugars and of dietary fibres within the large intestine by means of the bacterial flora of the intestine.

The production of short-chain fatty acids takes place along the entire large intestine with a gradient which decreases from the ileocaecal valve to the rectum. At the moment at which these short-chain fatty acids come into contact with the cells of the mucous membrane of the colon, they are rapidly captured within the cells where they are metabolized to acetyl-CoA, which is a fundamental factor of energy metabolism. Of the four short-chain fatty acids mentioned above, butyric acid is considered the most important source of energy for the colocytes since it is responsible for about 70% of their oxygen consumption. About 70-90% of all of the butyric acid produced in the colon is metabolized by the colocytes (Velazquez O. C. et al, Dietary Fiber in Health and Disease, Plenum Press, N.Y., 1977, 123-134; Wachtershauser A. et al., Eur. J. Nutr., 2000, 39, 164-171).

Short-chain fatty acids are considered to be the main source of energy for the cells of the mucous membrane of the colon and also to be fundamental factors in the control of the growth of the mucous membrane itself In fact a lack or substantial reduction thereof is often correlated with many functional disorders or organic pathological conditions such as, for example, disorders due to altered intestinal regularity, inflammatory intestinal conditions, ulcerative colitis, Crohn's disease, colonic neoplasia, etc. SCFAs and butyric acid or its salts in particular also intervene in the regulation of the proliferation of any colonic epithelial cells, not only favouring processes of re-epithelialization of the normal mucous membrane, but also inhibiting the proliferation of tumour cells, particularly by inhibition of the synthesis of the DNA of the tumour cell and by re-establishment of its natural apoptosis (Wachtershauser A. et al, Eur. J. Nutr., 2000, 39, 164-171).

Bearing in mind the key role played by butyric acid in the regulation of these extremely important biological activities of the colon, its administration, in conditions of absolute or relative deficit, represents an action of fundamental importance.

The endogenous production of butyric acid requires the presence of soluble dietary fibres which are fermented for this purpose by the bacterial flora of the colon. As well as being subject to fermentation by the bacterial flora and thus leading to the production of endogenous butyric acid, inulin in particular, amongst the soluble dietary fibres, is itself an important factor stimulating saprophytic bacterial growth, thus helping to promote bacterial colonization and to regulate the equilibrium of the bacterial flora of the intestine (Gibson, R. G. et al., Gastroenterology, 1995, 108, 975-982; Nyman M. Br. J. Nutr. 2002, 87, s163-168).

The supplementary supply of short-chain fatty acids and of fibre can therefore be considered a constant need, even for patients who do not show clear signs of disorders or pathological conditions at intestinal level, because of ever more frequent recourse to incorrect eating habits, to inappropriate dietary regimes, and to the use of ever more refined foodstuffs which are less and less rich in roughage and coarse fibre in particular.

In very many cases, in spite of the presence of a normal supply of fibre, the fermentation process itself may be deficient and may not lead to sufficient formation of butyric acid. This reduced or absent intestinal fermentation activity is, in most cases, caused by qualitative and quantitative modifications of the bacterial flora of the intestine which are due in turn to the ingestion of substances which inhibit the development and normal growth of the flora, such as antibacterial agents, preservatives, antibiotics, etc.

As a result of this general dietary-fermentary impoverishment, the production of butyric acid may therefore be reduced to levels such as not to supply adequate energy and protection to the intestine.

The breakdown of the delicate balance of interaction between exogenous factors (dietary fibre) and endogenous factors (bacterial flora) may therefore lead to the appearance of the above-mentioned organic or functional alterations affecting the intestine and, in particular, the colon.

In the presence of a reduced or insufficient colonic endolumenal concentration of butyric acid, at the moment, the most appropriate action is therefore the supply of a sufficient quantity of exogenous butyric acid, directly into the colon.

Currently, compositions based on butyric acid alone or on its Na⁺, Ca⁺⁺, and Mg⁺⁺ salts are available and the only route which can ensure that appropriate concentrations of that acid reach the interior of the colon is the rectal route which, however, since it does not enable the proximal part of the colon to be reached, limits the supply purely to the distal colon, with the understandable and considerable inconvenience connected with this administration route.

### DESCRIPTION OF THE INVENTION

Bearing in mind the impracticality of this latter administration route for supplementary dietary purposes and the need to limit the energy deficit and to re-establish intestinal balance, it has now surprisingly been found and constitutes the subject of the present invention that the combination of butyric acid itself, or of a salt thereof, with a soluble fibre such as, for example, inulin, in an oral formulation leads to a very significant synergic effect between the two components, leading to amplification of the effects that may be produced by the administration of the individual substances.

The combination according to the invention in fact leads to a synergy of the effects of the two substances which thus make up for the energy and protective deficit due to the lack or reduced production of endogenous butyric acid.

A subject of the present invention is therefore oral pharmaceutical or dietary compositions containing a short-chain fatty acid, in particular butyric acid, in combination with a soluble or water-dispersible dietary fibre.

The active components that are present in the mixture can be used in the most appropriate physical state for the production of a suitable form for administration; since the food supplement or the pharmaceutical composition is intended for oral administration, the preferred form is the solid form.

In order to produce these solid forms, in particular the tablet form, since butyric acid is a liquid, a solid salt of the acid such as, for example, calcium butyrate, sodium butyrate, or magnesium butyrate may be used, or the acid itself may be supported on a solid substrate of inert material by the known spray-dry technique or by adsorption.

As solid substrates, it is possible to use the excipients that are normally used for the preparation of tablets such as, for example, gum arabic, maize starch, pre-gelatinized starch, pectin, monosaccharide and polysaccharide sugars, alginates, microcrystalline cellulose, alkyl derivatives or hydroxyalkyl derivatives of cellulose with low, medium and high viscosity, monoprotic and polyprotic mineral salts, cyclodextrin, alkylcyclodextrin, hydroxyalkylcyclodextrin, pyrrolidones or derivatives, monocarboxylic organic salts and/or esters, polycarboxylic organic salts and/or esters, inorganic substrates such as colloidal silica, talc, and organic and inorganic ion-exchange resins. In order to produce a powder from a liquid, atomization is therefore performed by the drying of a suspension of liquid butyric acid and solid substrate by the spray-dry technique, or butyric acid is adsorbed on one of the above-mentioned substrates.

In both cases, a powder containing proportional quantities of butyric acid dispersed in the solid substrate is obtained.

In a preferred embodiment, the compositions of the invention are preferably formulated in a unitary-dose form for oral administration which can reach the specific colonic section of the intestine almost intact or in a manner such that most of the active ingredients reach the colon cavity directly, thus passing through the gastric intestinal portion and the first portion of the intestinal tract.

This requirement takes account of the fact that, when butyric acid or its salts are administered orally (foods, capsules, or plain tablets) they are absorbed very rapidly and completely by the small intestine to the extent that they do not manage to reach the colon.

This can be achieved by the use of controlled-release techniques which have their characteristic target site in the colonic section. These techniques are known in the pharmaceutical field and are normally used to formulate active substances of other types which require a specific release time and/or site such as, for example, intestinal anti-inflammatories (Brunner N. et al., Aliment. Pharmacol. Ther., 2003, 17, 395-402], systemic anti-inflammatories, anti-ulcerative agents, anti-microbial agents, or substances for energizing the mucous membrane.

European patent EP1183014, which is incorporated herein by reference, describes, for example, a multi-matrix controlled-release technique which is known by the trade mark MMX and is characterized by the dispersion of the active ingredient in a successive and progressive mixture of three different, interconnected matrices.

Other techniques which are theoretically suitable for the formulation of the composition of the invention are described in EP572942 and WO 00/28974, which are also incorporated herein by reference.

These techniques can bring about protection of the active ingredients throughout the transit through the stomach and during the passage through the first sections of the small intestine (the duodenum and the jejunum in particular) in order to release them directly in contact with the wall of the large intestine, precisely where their maximum concentration is required for an optimal effect.

These techniques are characterized by progressive and slow erosion of the tablet or other suitable solid form for the time necessary for the gastrointestinal transit, ensuring optimal and uniform distribution of the active ingredients along the entire mucous membrane of the colonic section.

It has thus been possible to provide a local topical treatment, utilizing to the maximum the energizing and protective capacities of butyric acid, which can thus act directly on the specific section of the mucous membrane of the colon, in combination with those of inulin which is thus brought into contact with the bacteria which can ferment it directly and produce further quantities of short-chain fatty acid.

On the basis of the foregoing, a further subject of the present invention is therefore controlled-release, gastro-resistant, oral pharmaceutical or dietary compositions containing a mixture of short-chain fatty acid and soluble fibre, which can pass through the entire gastric section and the first intestinal section without disintegrating and can release the active ingredients directly at colonic level.

The following examples are included to illustrate the invention further without being limiting thereof.

### EXAMPLES

### Example 1

3.075 kg of calcium butyrate (equal to 2.5 kg of butyric acid) was mixed with 1 kg of maize starch, 2.5 kg of inulin, 50 g of stearic acid, and 50 g of soya lecithin and mixed with water to a pasty consistency. The paste was then divided into granules by passing it through a drum granulator and, after the addition of 4 kg of maltodextrin, 1.975 kg of microcrystalline cellulose, 1 kg of medium viscosity sodium carboxymethyl cellulose, 200 g of colloidal silica, and 150 mg of magnesium stearate, was subjected to compression to a unit weight of 1400 mg/tablet (equivalent to a content per unit of 250 mg of butyric acid and 250 mg of inulin for each core).

The cores were then film-coated with an alcoholic suspension usable for depositing, per unit, on the cores, 20 mg of lac, 10 mg of talc, 6 mg of titanium dioxide, and 4 mg of triethyl citrate. The tablets thus produced were found to be capable of resisting disintegration in 0.1 N hydrochloric acid (simulating the gastric contents) for 1 hour or more and of progressively releasing the active ingredients contained, after an initial lag time, over the next 8 hours, in a buffered pH 7.4 solvent simulating the fluid that is present at intestinal level.

### Example 2

3.075 kg of calcium butyrate (equal to 2.5 kg of butyric acid) was mixed with 2.5 kg of inulin, with 300 g of sodium starch glycolate, 50 g of soya lecithin, and 950 g of microcrystalline cellulose, and mixed with 100 g of beeswax which had been heated to melting point, and then with water, to a pasty consistency. The paste was then divided into granules by passing it through a drum granulator and, after the addition of 4 kg of maltodextrin, 1.975 kg of dibasic calcium phosphate, 1 kg of medium-viscosity sodium carboxymethyl cellulose, 200 g of colloidal silica, and 150 mg of magnesium stearate, was subjected to compression to a unit weight equivalent to a content of 250 mg of butyric acid and 250 mg of inulin for each core.

The cores were then film-coated with an alcoholic solution of methacrylic acid and methacrylic esters, talc, triethyl citrate, and iron oxide which was able to deposit about 40 mg of coating per unit on the cores.

The tablets thus produced were found to be capable of resisting disintegration in 0.1 N hydrochloric acid (simulating the gastric contents) for 1 hour or more and of progressively releasing the active ingredients contained, after an initial lag time, over the next 8 hours, in a buffered pH 7.4 solvent simulating the fluid that is present at intestinal level.

### Example 3

2.5 kg of maltodextrin was added to and dispersed in 2.5 kg of butyric acid; the suspension, optionally diluted with water to the ideal consistency, was dried by atomization or spray-drying and a powder containing about 50% of butyric acid was obtained; 50 g of soya lecithin, 80 g of beeswax, and 950 g of lactose were added to this powder and mixed to a pasty consistency with a binding solution produced by dispersing 150 g of low-viscosity sodium carboxymethyl cellulose in 5 litres of water. The paste was then divided into granules by passing it through a drum granulator and, after the addition of 1.2 kg of microcrystalline cellulose, 0.6 kg of hydroxymethyl cellulose, 150 g of colloidal silica and 100 g of magnesium stearate, was subjected to compression to a unit weight equivalent to a content of 250 mg of butyric acid and 250 mg of inulin for each core.

The cores were then film-coated with an alcoholic solution of methacrylic acid and methacrylic esters, talc, triethyl citrate and iron oxide so as to deposit about 30 mg of coating per unit on the cores.

The tablets thus produced were found to be capable of resisting disintegration in 0.1N hydrochloric acid (simulating the gastric contents) for 1 hour or more and of progressively releasing the active ingredients contained, after an initial lag time, over the next 8 hours, in a buffered pH 7.4 solvent simulating the fluid that is present at intestinal level.

### Example 4

1.2 kg of pre-gelatinized starch and 2.4 kg of lactose were added to and dispersed in 2.4 kg of butyric acid; the suspension, optionally diluted with water to the ideal consistency, was dried by atomization or spray-drying and a powder containing about 40% of butyric acid was obtained; 30 g of soya lecithin, 20 g of sodium dioctylsulphosuccinate, 100 g of finely divided stearic acid, and 800 g of lactose were added to this powder and were mixed to a pasty consistency with a binding solution produced by dispersing 150 g of medium-viscosity hydroxypropylmethyl cellulose in 5 litres of water. The paste was then divided into granules by passing it through a perforated drum granulator and, after the addition of 1.2 kg of microcrystalline cellulose, a further 0.8 kg of hydroxypropylmethyl cellulose, 120 g of colloidal silica, and 100 g of magnesium stearate, was subjected to compression to a unit weight equivalent to a content of 250 mg of butyric acid and 250 mg of inulin for each core.

The cores were then film-coated with an aqueous dispersion of methacrylic acid and methacrylic esters, talc, triethyl citrate and titanium dioxide so as to deposit about 40 mg of film-forming coating per unit on the cores.

The tablets thus produced were found to be capable of resisting dissolution in 0.1N hydrochloric acid (simulating the gastric contents) for more than 1 hour and of progressively releasing the active ingredients contained, after an initial lag time, over the next 8 hours, in a buffered pH 7.4 solvent simulating the fluid that is present at intestinal level, in accordance with the following profile:
after 2 hours about 40% of the active ingredients
after 4 hours about 70% of the active ingredients present,
at 8 hours, more than 90% of the active ingredients had been given up.

The clinical study described below was performed with the tablets thus produced.

The study was performed on 18 adult patients of both sexes, divided into three homogeneous groups each of 6 patients, who were suffering from inflammatory bowel disease (IBD) and were treated as follows:

Group 1: 250 mg butyric acid

Group 2: 250 mg inulin

Group 3: 250 mg butyric acid+250 mg inulin

The active ingredients were transported in tablets which were indistinguishable in shape, size, weight and colour.

The treatments were administered orally daily before the main meal for a period of 4 weeks.

Before the start of the treatment (base time) and then upon completion of the 4 weeks of treatment, each patient was assessed for the presence of the symptom of diarrhoea.

This symptom was quantified, according to its intensity, on a subjective 5-point scale graduated from 0 (absence of indisposition) to 4 (indisposition of considerable gravity). The mean intensity at the base time and at the end of the period of treatment and the percentage of improvement relative to the base time were calculated for this symptom. The results obtained are given in Table 1 below:

TABLE 1
Effect of daily oral treatment for 4 weeks on the symptom of IBD

| | | | 250 mg butyric acid + |
|---|---|---|---|
| | 250 mg butyric acid | 250 mg inulin | 250 mg inulin |
| Symptom | (n = 6) | (n = 6) | (n = 6) |
| Diarrhoea | | | |
| base | 2.7 | 2.9 | 2.8 |
| final | 1.5 | 2.1 | 1.0 |
| % improvement | 44 | 27 | 64 |

### RESULTS

The table given above shows that the administration of tablets containing the combination of butyric acid (250 mg) and inulin (250 mg) brought about a 45% improvement in the symptom compared with the administration of butyric acid (250 mg) alone. Moreover, this percentage of improvement was much greater than the improvement provided by the administration of inulin alone which, as shown in the table, was 27%, i.e. corresponding to about half of that achieved with the mixture.

It was consequently shown that the combination of butyric acid and inulin leads to a synergic effect which is clear from the improvement of at least one symptom which is characteristic of the intestinal condition IBD (inflammatory bowel disease); this improvement is greater as a percentage than that which is achieved by the administration of the individual active ingredients.

What is claimed is:

1. An oral pharmaceutical or dietary composition containing at least one short-chain fatty acid or salt, ester and/or amide thereof, in combination with a complex sugar and/or dietary fibre in which the complex sugar and/or dietary fibre is selected from inulin, pectin, dextrin, maltodextrin or derivatives thereof and with one or more pharmacologically acceptable excipients.

2. The composition according to claim 1, wherein the short-chain fatty acid is a linear or branched C₁-C₅ monocarboxylic organic acid.

3. The composition according to claim 1, wherein the short-chain fatty acid is selected from the group consisting of acetic acid, propionic acid, butyric acid and isovaleric acid.

4. The composition according to claim 3, wherein the short-chain fatty acid is butyric acid.

5. The composition according to claim 1, wherein a quantity of from 5 to 50% by weight of the short-chain fatty acid is included.

6. The composition according to claim 5, wherein a quantity of from 10 to 30% by weight of the short-chain fatty acid is included.

7. The composition according to claim 1, wherein a quantity of from 5 to 50% by weight of the soluble dietary fibre is included.

8. The composition according to claim 7, wherein a quantity of from 10 to 30% by weight of the soluble dietary fibre is included.

9. The composition according to claim 1 in tablet, capsule, granule and/or micro-granule form.

10. The composition according to claim 1, wherein it is an intestinal controlled-release composition.

11. The composition according to claim 1, wherein it further contains a gastro-resistant coating.

12. A method for the treatment of intestinal disorders, inflammatory disorders, and pathological conditions of the intestinal mucous membrane and for the preventive or limiting treatment of intestinal neoplasia which comprises the administration to a patient in need of such a treatment of a pharmaceutical or dietary composition comprising a short-chain fatty acid in combination with a soluble dietary fibre in which the complex sugar and/or dietary fibre is selected from inulin, pectin, dextrin, maltodextrin or derivatives thereof.

13. The method according to claim 12, wherein the short-chain fatty acid is a linear or branched C₁-C₅ monocarboxylic organic acid.

14. The method according to claim 12, wherein the short-chain fatty acid is selected from the group consisting of acetic acid, propionic acid, butyric acid and isovaleric acid.

15. The method according to claim 14, wherein the short-chain fatty acid is butyric acid.

16. The method according to claim 12, wherein a quantity of from 5 to 50% by weight of the short-chain fatty acid is included.

17. The method according to claim 16, wherein a quantity of from 10 to 30% by weight of the short-chain fatty acid is included.

18. The method according to claim 12, wherein a quantity of from 5 to 50% by weight of the soluble dietary fibre is included.

19. The method according to claim 18, wherein a quantity of from 10 to 30% by weight of the soluble dietary fibre is included.

20. The method according to claim 12, wherein the composition is in tablet, capsule, granule and/or micro-granule form.

21. The method according to claim 12, wherein the composition is an intestinal controlled release.

22. The method according to claim 12, wherein the composition further includes a gastro-resistant coating.
